(19) United States
(12) Patent Application Publication
(10) Pub. No.: US 2023/0242970 A1
(43) Pub. Date: Aug. 3, 2023

(54) Detection of Chemicals and Molecules Using Cell-free Biosensor Lateral Flow Assays

(71) Applicant: Stemloop, Inc., Evanston, IL (US)

(72) Inventors: Khalid Kamal Alam, Morton Grove, IL (US); R Cooper Baer, Chicago, IL (US)

(21) Appl. No.: 18/160,468

(22) Filed: Jan. 27, 2023

Related U.S. Application Data
(60) Provisional application No. 63/304,257, filed on Jan. 28, 2022.

(51) Int. Cl. C12Q 1/6806; C12Q 1/6869; C12Q 1/6825

(52) U.S. Cl. CPC C12Q 1/6806; C12Q 1/6869; C12Q 1/6825

(57) ABSTRACT

Provided herein is a cell-free biosensor lateral flow device kit for detection of analytes of interest and methods of using thereof. The device comprises a substrate and a first end, wherein the first end comprises a sample loading portion. The device additionally may comprise a sensor module, wherein the sensor module comprises an allosteric transcription factor regulated in vitro transcription reaction, and a transduction module, wherein the transduction module comprises Bait and Prey nucleic acids which sense the output of the sensor element and a reporter conjugate which accumulates at a test zone when an analyte the sensor element senses is present in the sample.

### CROSS REFERENCE TO RELATED APPLICATIONS

This application claims the benefit of and priority to U.S. Provisional Patent Application No. 63/304,257, filed on Jan. 28, 2022, the contents of which are incorporated by reference herein in its entirety.

### SEQUENCE LISTING

This application contains a sequence listing filed in electronic form as an xml file entitled STEM-0100US_ST26.xml, created on Jan. 26, 2023, and having a size of 9,182 bytes. The content of the sequence listing is incorporated herein in its entirety.

### TECHNICAL FIELD

The subject matter disclosed herein is generally directed to cell-free biosensor lateral flow assays for the detection of chemical and molecular analytes of interest.

### BACKGROUND

Chemical and molecular detection techniques are important for understanding the presence, absence, or amount of an analyte of interest in a given sample. These analytes can include toxins, pollutants, contaminants, drugs, pathogens, and biomarkers, and are measured in samples as varied as consumable goods (e.g., food and drink), the environment (e.g., air, water, soil), and specimens from animals (e.g., blood, urine, saliva), plants, or other organisms. Additionally, detection of chemical and molecular analytes plays a central role in informing and managing various engineering, manufacturing, and industrial processes for the determination of yield, quality, safety, and compliance.

Detection of chemicals and molecules is traditionally performed within laboratories capable of analytical techniques. While the specific method depends on the sample and analyte of interest, they often rely on sophisticated equipment to analyze samples, such as gas chromatography—mass spectrometry and inductively coupled plasma mass spectrometry (Beale et al., 2018; Mittal et al., 2017). These techniques can be highly quantitative and reliable; however, they are typically costly, complicated, and require days or longer to turnaround samples from the time of collection and provide results. Additionally, due to the inherent infrastructure requirements of analytical laboratories, they are typically centralized and require that samples are appropriately preserved and transported to the laboratory, thereby increasing cost, complexity, and time-to-result. These characteristics of laboratory testing have spurred development of new chemical and molecular detection methods that provide results at lower cost, that function directly at the point-of-use, are easy to use by laypersons, and offer quick turnaround.

Biological sensors (biosensors) are an alternative to laboratory-based chemical and molecular detection methods due to their potential for lower cost, ease-of-use, and results at the point-of-use. Biosensors use biological molecules, such as antibodies and enzymes, that transduce the binding of a chemical or molecule of interest into an observable signal. Antibodies, for example, are a type of biosensor that are capable of binding to a specific analyte of interest (known as an antigen) and forming an antibody:antigen complex. When an antibody is conjugated to a detection agent, such as a gold nanoparticle, the resulting complex can be captured and visually detected on simple, low-cost, and rapid lateral flow devices [https://doi.org/10.1016/j.trac.2016.06.006]. Common examples of lateral flow tests that utilize antibodies include at-home pregnancy tests, which detect Human Chorionic Gonadotropin in urine samples [https://doi.org/10.1016/j.trac.2015.10.017], and at-home COVID tests, which detect SARS-CoV-2 viral particles in biological samples as an indicator of infection [https://doi.org/10.1016/j.trac.2021.116452]. Enzymes offer yet another example of a biosensor. Glucose oxidase, for example, is an enzyme used in blood glucose monitoring devices for diabetes management [https://doi.org/10.1021/cr068123a]. Similarly, the enzyme lactate oxidase has been repurposed for lactate monitoring [https://doi.org/10.1016/j.bbrep.2015.11.010]. In both these cases, the analyte of interest serves as the substrate for the respective enzyme, and the resulting product(s) of enzyme catalysis can be observed and measured to determine the presence, absence, or concentration of the analyte.

Recently, a class of sensors known as “cell-free biosensors” (CFBs) has been developed. CFBs are characterized by their use of cell-free gene expression reactions that support the transcription and translation (“expression”) of genes encoded in DNA [https://doi.org/10.1038/s41576-019-0186-3, https://doi.org/10.1038/s43586-021-00046-x, https://doi.org/10.1007/978-3-030-23217-7_130, https://doi.org/10.1016/j.cobme.2019.08.005, https://doi.org/10.1002/biot.202000187, https://doi.org/10.3390/bios12050318]. These gene expression systems are termed “cell-free” because, while they rely on cellular components, they do not utilize intact, membrane-bound cells. In CFBs, a reporter gene is produced (“expressed”) as a function of whether an analyte of interest is present in the sample. Expression of the reporter gene results in the synthesis of an RNA or protein molecule that can be readily observed and measured, such as a fluorescence activating RNA, fluorescent protein, or colorimetric enzyme [https://doi.org/10.1038/s41587-020-0571-7, https://doi.org/10.1038/s41587-020-0571-7, https://doi.org/10.1021/acssynbio.9b00388, https://doi.org/10.1021/acssynbio.9b00348]. Expression in CFBs is controlled by allosteric transcription factors (aTFs), which regulate gene expression by binding to a DNA template and either preventing or enabling RNA polymerase to initiate transcription [https://doi.org/10.1016/j.copbio.2021.01.008]. Thus, the reporter gene is only produced if the analyte of interest in present in the sample and transcription is allowed to proceed. A benefit of using aTFs for chemical and molecular sensing is their ability to distinguish between isomers or closely related chemical species that differ by a single functional group [https://doi.org/10.1038/s41589-022-01072-w, https://doi.org/10.1021/acssynbio. 1c00402], which can be difficult to accomplish using standard analytical chemistry. Furthermore, the use of gene expression systems inherently enables signal amplification and the use of genetic circuits [https://doi.org/10.1073/pnas.2111450118]. Whole-cell biosensors, which utilize intact, living, and membrane-bound cells for sensing and detection, also utilize aTFs and gene expression processes [https://doi.org/10.1016/j.bios.2021.113359]. However, cell-based technologies are inherently subject to the regulatory concerns surrounding biocontainment or the use of genetically modified organisms. Unlike whole-cell biosensors, CFBs are not subject to evolutionary pressures that can challenge the functionality, stability, and robustness of whole-cell biosensor technology. CFBs also do not require active or passive transport of the analyte across the cell membrane.

Cell-free biosensors offer relevant sensitivity and specificity for an analyte of interest and are a compelling alternative to traditional analytical chemistry. Compared to laboratory approaches, cell-free biosensors are lightweight, inexpensive, easy-to-use by laypersons, and neither use nor generate hazardous wastes. Furthermore, cell-free biosensors can be readily stabilized and preserved through lyophilization, also known as freeze-drying, and therefore can be stored and distributed without requiring cold-chain [https://doi.org/10.1016/j.bej.2018.07.008]. When lyophilized, CFBs can be activated through simple rehydration of the system, making it compelling for detection of analytes in aqueous solutions. For this reason, CFBs are increasingly being investigated for water quality monitoring applications, where sampling and detection can be performed by merely adding a drop of sample water directly to the CFB [https://doi.org/10.1038/s41587-020-0571-7, https://doi.org/10.1038/s41545-020-0064-8]. These features make CFBs amenable for point-of-use applications, such as at-home testing. Due to these features, CFBs have been developed for several targets including pharmaceuticals, pesticides, personal care products, metabolites, ions and metals.

RNA Output Sensors Activated by Ligand Induction (ROSALIND) is one such cell-free biosensing approach [https://doi.org/10.1038/s41587-020-0571-7, https://doi.org/10.1038/s41587-020-0571-7, https://doi.org/10.1007/978-1-0716-1998-8_20]. ROSALIND leverages allosteric transcription factors (aTFs) in an in vitro transcription reaction to rapidly produce visible, fluorescent outputs in response to the detection of an analyte of interest. ROSALIND has been used for sensitive and specific assays for broad classes of compounds including heavy metals, halogen anions, antibiotics, and pesticides. In ROSALIND, a double stranded DNA linear transcription template containing a T7 promoter and a downstream operator sequence for aTF binding controls the expression of a gene encoding a fluorescence activating RNA. ROSALIND has several advantages over other cell-free biosensor approaches in that it consists of a few, well-defined purified components (rather than poorly-defined cellular lysates) and does not require the additional step of protein synthesis through translation. ROSALIND is therefore highly robust, predictable, reproducible, and eliminates the resource and time-intensive step of translation that protein-based reporters require (Jung & Alam et al., 2020).

Despite recent progress, existing CFBs have several limitations. Fluorescence and absorbance-based colorimetric outputs can require LEDs, optical filters, photoresistors, and other electronic hardware and software for detection. These equipment requirements increase the cost and complexity of CFBs and limit their use by laypersons for point-of-use detection. In addition, CFBs developed to date have generally required temperature-controlled incubation at an elevated temperature, often between 30 and 37° C. (Jung et al., 2020; Silverman et al., 2020). In addition to the expense of heating and cooling elements, the incubation requirement adds significant power demands to such sensing systems. A system that can be used without electronics and with visualization by the naked eye would be a desirable improvement over the existing state of the art. For example, recent innovations in CRISPR-Cas based diagnostics have coupled Cas protein detection of target nucleic acids to nucleic acid lateral flow tests [https://doi.org/10.1038/s41587-020-0513-4, https://doi.org/10.1038/s41551-020-00603-x]. These approaches, coupling highly specific nucleic acid sensing Cas enzymes to simple lateral flow tests, decrease or eliminate the complexity of testing for pathogens and could be extended to other biosensing detection modalities.

Here, Applicants demonstrate that cell-free biosensor reactions, specifically ROSALIND, can be coupled with lateral flow technology to create a simple, low-cost assay for chemical and molecular detection. In this approach, the cell-free gene expression system produces an RNA in response the presence of an analyte of interest. When the RNA is expressed, it is capable of detection on lateral flow devices within minutes and without necessitating additional equipment or electronics.

Citation or identification of any document in this application is not an admission that such a document is available as prior art to the present invention.

### SUMMARY

Cell-free biosensor lateral flow device kits for the detection of one or more analytes of interest are provided comprising: a sensor reaction module comprising reagents for an in vitro transcription reaction that is conditionally activated by the presence of the analyte of interest to thereby produce a transcript; a lateral flow device comprising a substrate with a sample deposition zone, a first capture region comprising a first binding agent, and a second capture region comprising a second binding agent; and a transduction module comprising oligonucleotides and a detectable reporter to interface the sensor reaction module with the lateral flow device.

One or more components of the transduction module can be present or immobilized on the substrate, which may optionally be a flexible paper substrate.

The cell-free biosensor reaction (“sensor reaction module”) can comprise a nucleic acid template; an allosteric transcription factor (aTF) capable of binding the analyte of interest; a buffer system enabling transcription including nucleotide triphosphates, buffers, salts, reducing agents, and accessory proteins; and a nucleic acid polymerase, or any combination thereof. In an aspect, the nucleic acid template includes a promoter sequence for binding and transcription initiation by a nucleic acid polymerase, an operator sequence to which the aTF is capable of reversibly binding as a function of binding to the analyte of interest, and encodes a “Trap” sequence capable of direct interaction with a transduction module.

The transduction module can further comprise a “Bait” nucleic acid, a “Prey” nucleic acid, and a detectable reporter. In an aspect, the Trap nucleic acid template encodes a polynucleotide comprising a 5′ end with reverse complementarity to the Prey nucleic acid and a 3′ end complementary to the Bait nucleic acid. In an embodiment, the Bait nucleic acid is a single stranded nucleic acid, which may comprise at least 50% sequence similarity up to 100% sequence identity to 5′-ACTACCGTCAGCATTATGTGAGTGAAACAA-3′ (SEQ ID NO: 1). In an aspect, the Bait nucleic acid is between 25 and 50 nucleotides long. The Bait nucleic acid can comprise a chemical moiety, optionally the chemical moiety is biotin.

The Prey nucleic acid can comprise a single stranded nucleic acid. In an aspect, the Prey nucleic acid may comprise at least 50% sequence similarity up to 100% sequence identity to 5′-AGTGATATTGCCACCGACCTCAATCAATAA-3′ (SEQ ID NO: 2). In an aspect, the Prey nucleic acid is between 25 and 50 nucleotides long. The Prey nucleic acid can comprise a chemical moiety, optionally the chemical moiety is fluorescein.

In an aspect, the detectable reporter comprises an antibody-reporter conjugate. The antibody-reporter conjugate can comprise an antibody that binds to a chemical moiety attached to the Prey nucleic acid conjugated to the detectable reporter. In an aspect, the antibody is an anti-fluorescein isothiocyanate (FITC) antibody. The detectable reporter can comprise a gold or latex nanoparticle, or a colorimetric enzyme such as horseradish peroxidase, beta-galactosidase, or catecholase.

In one embodiment, the in vitro transcription reagents comprise a linear or circular double stranded DNA molecule template to which the Trap nucleic acid is encoded.

In an embodiment, the nucleic acid polymerase is a DNA-dependent RNA polymerase.

The RNA polymerase can be from a bacteriophage or a bacterium. In an aspect, the RNA polymerase consists of a single sub-unit. In one embodiment, the RNA polymerase is T7 RNA polymerase, SP6 RNA polymerase, Syn5 RNA polymerase, KP34 RNA polymerase, VSW-3 RNA polymerase, SPbeta RNA polymerase, or T3 RNA polymerase. The RNA polymerase may be E. coli RNA polymerase.

In one embodiment, the sensor module comprises one or more nucleic acids, and the transcript produced has complementarity to one or more of the nucleic acids of the transduction module.

In an embodiment, the allosteric transcription factor regulates the transcription reaction in response to the binding of the analyte of interest. The allosteric transcription factor can comprise a member of the AraC, AsnC/Lrp, Crp-Fnr, Fur, CadC/ArsR, Ic1R, TetR, Lad, MerR, CsoR, MarR or functionally similar families. In an embodiment, the allosteric transcription factor is engineered to alter its ligand sensitivity and/or specificity or other intrinsic physical property. In an aspect, the allosteric transcription factor is regulated by a chemical or element in the sample.

In an embodiment, the sensor module is applied to the sample deposition zone. The sensor module may be lyophilized, optionally the sensor module is lyophilized in the sample deposition zone of the substrate.

The analyte of interest may be a metal, including, but not limited to, lead, cadmium, mercury, zinc, copper, manganese, chromium, cobalt, nickel, antimony, or thallium. The analyte may be a pesticide or pesticide metabolite, including, but not limited to, atrazine, paraquat, a pyrethroid insecticide, 1,3,-dichloropropene, or an organophosphate. The analyte may be an environmental contaminant.

The lateral flow device of any of the previous claims, wherein the analyte is a Perfluorooctane sulfonic acid (PFOS) or Perfluorooctanoic acid (PFOA) or another perfluorinated compound, a polychlorinated biphenyl, a dioxin, a bisphenol, or a phthalate. The analyte may comprise an antibiotic or metabolite thereof. The analyte may be a tetracycline, an aminoglycoside, a carbapenem, a cephalosporin, a sulfonamide, a macrolide, a glycopeptide, a lincomycin, a penicillin, or a quinolone, or a metabolite thereof.

The sample to be tested can be water, including a drinking water sample from a home, educational facility, business, or other public or private place; water from an environmental source like a river, lake, pond, or groundwater; wastewater from a municipality, community, home, educational facility, factory, or other business or building. The sample to be tested can be from an industrial biotechnology source. In an aspect, the sample is culture supernatant, cell lysate, or other input or output product from a metabolic engineering experiment or process.

The sensor module may comprise two or more allosteric transcription factors. In an embodiment, the first and second aTF bind to different target chemicals. In an embodiment, the first and second aTF bind to the same or related target chemicals. In an embodiment, the two or more aTFs bind to the same operator on the template. The aTFs may bind to a different operator on the template.

Kits comprising the lateral flow devices of the present invention are provided herein.

Methods for detecting an analyte of interest in a sample, comprising contacting the sample with the sensor module and then transferring the reaction to the sample loading zone of the lateral flow device according to the present invention, wherein the sample flows from the sample loading portion of the substrate towards the first and second capture regions and generates a detectable signal are also provided.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Depiction of an exemplary embodiment of a cell-free biosensor reaction with outputs reported on a lateral flow device. A sample (left) is added to a tube containing a lyophilized bead of a cell-free biosensor reaction. Once rehydrated by the sample, the cell-free biosensor reaction is activated (center). The cell-free biosensor reaction comprises a regulated in vitro transcription reaction with an RNA polymerase, a DNA template encoding a Trap RNA sequence, and an allosteric transcription factor (aTF). In the absence of the analyte of interest, the aTF is bound to the DNA template and blocks transcription by RNA polymerase, resulting in no Trap RNA being produced. In the presence of the analyte of interest, the aTF changes conformation to preferentially bind to the analyte of interest over the DNA template, thereby allowing transcription of the DNA template by RNA polymerase to proceed and resulting in the production of the Trap RNA. When the sensor reaction is run on a lateral flow device (right), the absence of Trap RNA results in the formation of a single, visible control band (a negative test, top right). Whereas, in the presence of Trap RNA, a test and a control band are both visibly formed (a positive test, bottom right).

FIG. 2—Depiction of an exemplary embodiment of the lateral flow device with an embedded transduction module. After contact with the cell-free biosensor reaction, samples are added to the device on the sample pad and flow laterally through the conjugate pad and the nitrocellulose membrane towards the absorbent pad on the other end of the device. The conjugate pad contains the transduction module and includes a “Bait” oligonucleotide with a 5′ Biotin, a “Prey” oligonucleotide with a 3′ Fluorescein, and a “Reporter” gold nanoparticle conjugated to an anti-FITC antibody. The nitrocellulose membrane contains two capture regions including an upstream “Test Region” and a downstream “Control Region.” The test region contains streptavidin, which is capable of binding to the biotin moiety of the Bait oligonucleotide. The control region contains a secondary antibody that is capable of binding to the anti-FITC antibody.

FIGS. 3A-3B—Depiction of lateral flow test results from an exemplary embodiment of the cell-free biosensor lateral flow assay. The cell-free biosensor lateral flow assay can result in two scenarios. (FIG. 3A) In the first scenario—a negative (non-detect) test result in which Trap is not produced—the reagents flow past the test region and only the Bait oligonucleotide is captured through the binding interaction of the 5′ Biotin moiety of the Bait oligonucleotide and the streptavidin on the membrane. The reporter molecule continues to flow past the test region until it is captured in the second control region through the binding interaction of the anti-FITC antibody and the secondary antibody on the membrane. Accumulation of the reporter in the control region results in the formation of a visible band. Prey flows across the device and is not captured on the membrane. (FIG. 3B) In the second scenario—a positive detection result—the presence of the Trap RNA in the sample forms a ternary complex with the Bait and Prey oligonucleotides. The ternary complex is captured in the test region, due to the interaction of the 5′ biotin on the Bait, and the reporter aggregates in the test region due to the interaction between the anti-FITC antibody and the 3′ Fluorescein moiety on the Prey oligonucleotide. Excess reporter is captured downstream in the control region by binding to the secondary antibody. Accumulation of the reporter molecule in both the test and control regions results in the formation of two visible bands.

FIG. 4—Depiction of an exemplary embodiment of the DNA template for the cell-free biosensor. The DNA transcription template is depicted in Synthetic Biology Open Language (SBOL) visual specification (https://doi.org/10.1515/jib-2021-0013) and the top-strand DNA sequence in 5′ to 3′ orientation below. The SBOL visual depicts a DNA template with (from 5′ to 3′) a promoter for RNA polymerase, an operator sequence for allosteric transcription factor (aTF) binding, a Trap sequence, and a transcription terminator. The DNA sequence depicts an embodiment of the template for lead detection using the CadC aTF. The sequence includes a promoter for T7 RNA polymerase, the cadO operator sequence for CadC binding, the Trap sequence, and a T7 transcription terminator (SEQ ID NO: 3).

FIGS. 5A-5D—Validation and optimization of a transduction module according to an exemplary embodiment using a single stranded Trap DNA. (FIG. 5A) Picture of lateral flow test strips exposed to varying combinations of 1 μM Bait (B), Prey (P), and Trap (T) oligos as ssDNA. (FIG. 5B) Normalized test band density of strips from panel A measured by densitometry. (FIG. 5C) Comparison of optimized versus unoptimized Bait and Prey oligo concentrations in the detection of 1 nM ssDNA Trap oligo. (FIG. 5D) Titrations of Trap ssDNA oligo into optimized and unoptimized conditions.

FIG. 6—Further validation of a transduction module according to an exemplary embodiment using a purified Trap RNA. A purified Trap RNA was used to validate the transduction module with a lateral flow assay. Conditions included a “No Trap” (negative control) and 1 nM of a ssDNA Trap (positive control). Purified Trap RNA was added at three different concentrations and, after contact with the lateral flow strip, the results were quantified and measured for relative band intensity.

FIG. 7—Further validation of a transduction module according to an exemplary embodiment using a purified Trap RNA with thermal renaturation. Purified Trap RNA was thermally renatured and used to validate the transduction module with a lateral flow assay. Conditions included a “No Trap” (negative control) and 1 nM of a ssDNA Trap (positive control). Purified Trap RNA was added at two different concentrations and with or without thermal renaturation. After contact with the lateral flow strip, the results were quantified and measured for relative band intensity.

FIGS. 8A-8G—Validation of a sensor for lead detection according to an exemplary embodiment. (FIG. 8A) Time course of repressed and unrepressed transcription of Trap RNA under the control of the CadC-regulated cadO operator sequence at 37° C. (FIG. 8B) Lead-induced de-repression of Trap oligo transcription at room temperature. (FIG. 8C) Detecting transcription of Trap RNA at room temperature (˜22° C.) in the absence of CadC. (FIG. 8D) Lead-induced de-repression of Trap oligo transcription at room temperature for 10 minutes. (FIG. 8E) Optimization of Bait oligo concentration to reduce background signal in repressed but uninduced in vitro transcription (IVT). (FIG. 8F) Time course of repressed and derepressed IVT at room temperature. (FIG. 8G) Detection of 10 parts per billion (ppb) lead.

FIGS. 9A-9D—Validation of a lyophilized device embodiment that allows simple rehydration of the sensor module before use. (FIG. 9A) Picture of lyophilized sensor module in a microcentrifuge tube. (FIG. 9B) Results of lyophilized sensor rehydrated with water containing denoted concentration of lead and applied to transduction module at indicated time points. (FIG. 9C) Schematic showing process of lyophilizing sensor on paper disks prior to rehydration and application to LFA strips. (FIG. 9D) Results of paper-lyophilized sensor rehydrated with water containing denoted concentration of lead.

FIGS. 10A-10B—Optimization and fine-tuning the sensitivity of a cell-free biosensor lateral flow assay for the detection of lead according to an exemplary embodiment. (FIG. 10A) DNA template concentration in the sensor module was varied to optimize for the detection of 5 parts per billion (ppb) lead using the cell-free biosensor lateral flow assay, including photos of the resulting lateral flow strips and quantification through densitometry analysis. (FIG. 10B) Reducing assay concentration by increasing the volume of sample added to the sensor module (while keeping other assay component amounts fixed) leads to reduced sensitivity of the cell-free biosensor lateral flow assay at 1 part per billion lead.

FIGS. 11A-11B—Extensibility of the cell-free biosensor lateral flow assay for the detection of other analytes according to an exemplary embodiment. (FIG. 11A) A copper cell-free biosensor lateral flow assay was developed and validated using the CsoR allosteric transcription factor (aTF) and an appropriately designed DNA template containing the csoO operator sequence. Similarly, (FIG. 11B) a tetracyline cell-free biosensor assay was developed and validated using the TetR aTF and an appropriately designed DNA template containing the tetO operator sequence. aTC=anhydrotetracycline.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F.M. Ausubel et al. eds.); the series Methods in Enzymology (Academic Press, Inc.): PCR 2: A Practical Approach (1995) (M.J. MacPherson, B.D. Hames, and G.R. Taylor eds.): Antibodies, A Laboratory Manual (1988) (Harlow and Lane, eds.): Antibodies A Laboratory Manual, 2nd edition 2013 (E.A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

As used herein, an “environmental sample” may be a crude sample, which has not been purified or further manipulated prior to testing, such as water, soil, or a surface sample. The environmental sample may be a water sample. Water samples may be from a drinking water sample from a home, educational facility, business, or other public or private place. The water sample may be an environmental source like a river, lake, pond, or groundwater, In an example embodiment, the water is from a containment pond, such as an ash pond. The environmental sample may be from an industrial biotechnology source, or is a culture supernatant, cell lysate, or other input or output product from metabolic engineering.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to International Patent Publication WO 2020097610 and International Patent Publication WO 2020220031 and International Patent Publication WO 2022183102, incorporated herein by reference.

All publications, published patent documents, and patent applications cited herein are hereby incorporated by reference to the same extent as though each individual publication, published patent document, or patent application was specifically and individually indicated as being incorporated by reference.

### Overview

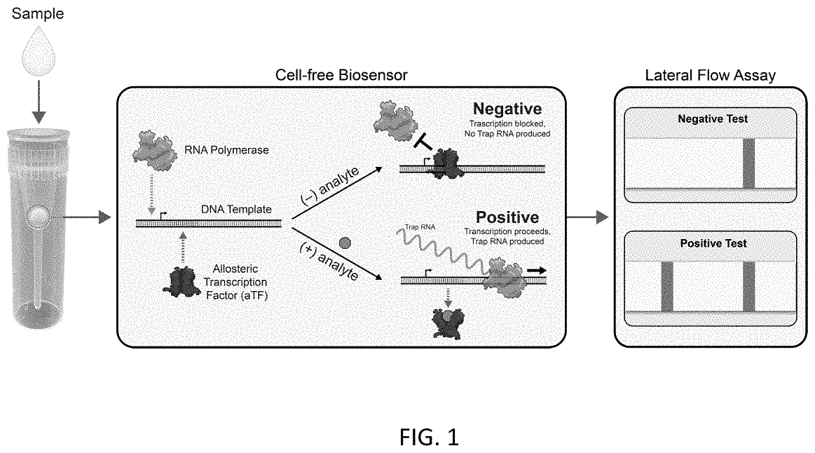

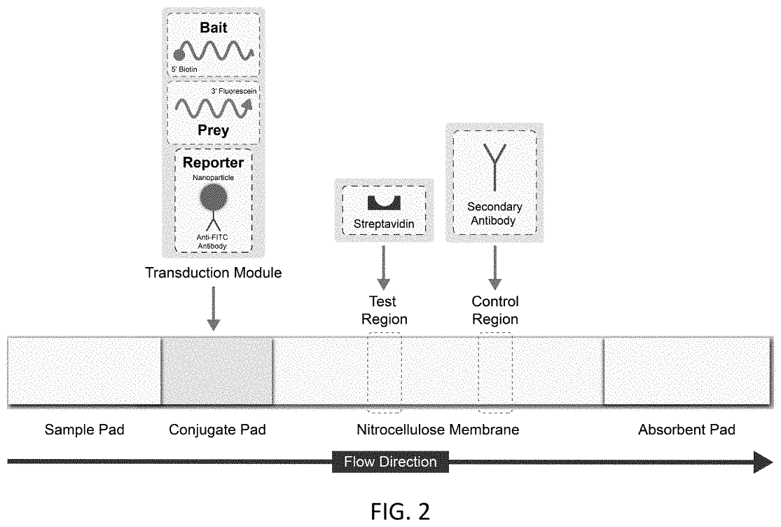

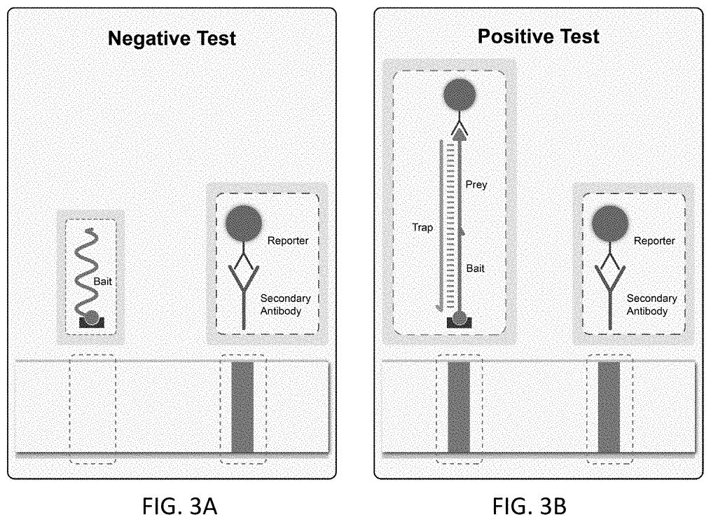

Embodiments disclosed herein provide cell-free biosensor lateral flow assay devices and methods for the detection of analytes of interest, including assays capable of detecting regulation-relevant concentrations of analytes of interest, run at room temperature or at an elevated temperature, and without requirement of heating elements, power, or additional spectroscopic means for reading results. In an embodiment, the system comprises a cell-free biosensor comprising reagents for an in-vitro transcription (IVT) reaction in which an allosteric transcription factor (aTF) regulates a promoter-operator for an RNA polymerase (FIG. 1). In the presence of a ligand of interest, the aTF alters its interaction with the promoter-operator region and either allows or prevents transcription of an RNA, referred to herein as a Trap RNA (“Trap”). Trap is designed to associate with a “Bait” oligonucleotide and a “Prey” oligonucleotide, which facilitates binding at a capture or “Test” region of the lateral flow device substrate and generating a visible output (FIGS. 2, 3). Moreover, the embodiments disclosed herein can be prepared in a lyophilized (“freeze-dried”) format for convenient distribution and point-of-use (POU) applications. The assays and methods find use in a variety of applications, including, but not limited to, testing of heavy metals, halogen anions, antibiotics, and pesticides.

Components of the system according to the present disclosure may be freeze-dried to the lateral flow substrate and packaged as a ready to use device, or the one or more modules may be added to the reagent portion of the lateral flow substrate at the time of using the device. After the samples are exposed to the sensor modules, samples to be screened are loaded at the sample loading portion of the lateral flow substrate. The samples are liquid samples or samples dissolved in an appropriate solvent, usually aqueous. The liquid sample reconstitutes the freeze-dried reagents of the sensor module and transduction module such that a detection reaction can occur. The liquid sample begins to flow from the sample portion of the substrate towards the first and second capture zones (FIG. 2). If target chemical(s) are present in the sample, the in vitro transcription reaction of the sensor reaction module is conditionally activated, thereby generating an RNA “Trap.” As the Trap RNA comes into contact with the Bait nucleic acid and Prey nucleic acid, the Trap RNA forms a bridge between the Bait and Prey nucleic acids, accumulating Prey nucleic acid that is captured at a first capture region by binding of the Bait nucleic acid to a first binding agent (FIG. 3). Detectable reporter of the transduction module is designed to bind at a second capture region and will bind irrespective of presence of the presence of analyte of interest and can provide a control for the reaction. Accordingly, if the target chemical(s) is not present in the sample, a detectable signal will appear at the second capture region, and if the target chemical(s) is present in the sample, a detectable signal will also appear at the location of the first capture region. Additional capture regions can be provided for detection of additional Trap RNAs, and therefore detection of a plurality of analytes of interest.

### Cell-Free Biosensor Lateral Flow Assay Kit

The lateral flow device comprises a substrate which comprises capture regions, each capture region comprising a binding agent. The lateral flow device includes a sample pad, a conjugate pad that contains reagents, and an absorbant pad. A sensor reaction module, a transduction module are provided, and can be added to the lateral flow device at an input portion of the substrate, typically on one end of the lateral flow substrate. In an aspect, one or more of the modules are provided in freeze-dried format on the lateral flow substrate in a defined reagent portion of the lateral flow substrate, typically on one end of the lateral flow substrate. The first capture region can comprise a test area and a second capture region can comprise a control area. A sample deposition zone of the substrate may be equivalent to, continuous with, or adjacent to the reagent portion comprising one or more modules of the device (FIG. 2).

In an embodiment, the lateral flow device further comprises a control module, which may comprise a control reporter that can bind to a control capture region, e.g., a first capture region. In an aspect, the control module comprises a detectable reporter molecule that is the same as the detectable reporter molecule of the transduction module, and thus may be considered subsumed by the detectable reporter molecule provided in the transduction module of the assay. In another aspect, the control module comprises a different detectable reporter molecule, i.e., a second detectable reporter molecule. The control capture region is designed and capable of binding the selected detectable reporter molecule.

### Sensor Reaction Module

The sensor reaction module can comprise reagents for an in vitro transcription reaction and a reaction template comprising a promoter sequence for a nucleic acid polymerase, a binding site for an allosteric transcription factor (an “operator” sequence) and an oligonucleotide encoding the Trap RNA is provided either in a first region of the substrate, for example the sample loading zone, or provided in an ampoule, tube, tray, plate, or other individual discrete volume for use with the assay (FIG. 1).

In an embodiment, the nucleic acid polymerase is a DNA-dependent RNA polymerase. The RNA polymerase can be from a bacteriophage or a bacterium. In an aspect, the RNA polymerase consists of a single sub-unit. In one embodiment the RNA polymerase is T7 RNA polymerase, SP6 RNA polymerase, Syn5 RNA polymerase, KP34 RNA polymerase, SPbeta RNA polymerase, VSW-3 RNA polymerase or T3 RNA polymerase. The RNA polymerase may be E. coli RNA polymerase holoenzyme. Depending on the RNA polymerase used, the reaction template, e.g., Trap nucleic acid template, can be configured to contain a promoter for the RNA polymerase. For example, when the RNA polymerase is a T7 polymerase, the promoter on the template can be a T7 RNA polymerase promoter.

### Reaction Template

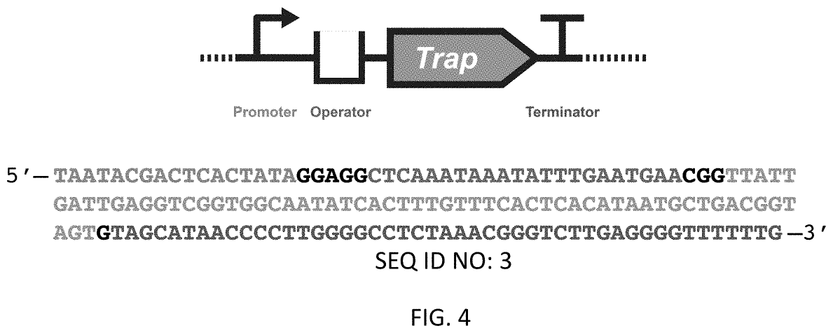

The reaction template, which may be a Trap nucleic acid template, comprises a promoter, an operator, and a nucleic acid molecule comprising a portion complementary to sequence with reverse complementarity to a Bait oligonucleotide on its 3′ end and a sequence complementary to a Prey oligonucleotide on the 5′ end (FIG. 4). In an aspect, the reaction template may optionally include a transcription termination sequence. In an embodiment, the reaction template is linear or circular double stranded DNA molecule, such that an in vitro transcription reaction produces a Trap nucleic acid, for example, an RNA product that is produced with complementarity to each of the nucleic acids that make up the transduction module. The Trap RNA is designed such that hairpins within the Trap RNA and homodimers of the fully transcribed sequence are unlikely to form. As the sequence immediately downstream of the promoter may also be transcribed and the template may contain a transcription termination sequence, special consideration is required to ensure the fully transcribed product is unlikely to interfere with the Trap portion of the RNA sequence or form homodimers with itself.

The operator sequence is selected to which an allosteric transcription factor (aTF) is capable of binding. In an aspect, the aTF is bound to the operator and, upon presence of a ligand in the sample, the aTF releases from the operator. In an aspect, the aTF binds to the operator upon presence of a ligand in the sample and transcription of the template is reduced or abolished. The promoter of the reaction template is selected for the particular RNA polymerase chosen for the reaction template, as detailed elsewhere herein. In an aspect, the operator sequence is downstream of the promoter and upstream of the trap sequence (FIG. 4).

A buffer system may further be provided with the sensor module, which may enable transcription. In an example embodiment, the buffer system is lyophilized together with the sensor module. In an aspect, the buffer system may include nucleotide triphosphates, for example, ribonucleotide triphosphates. Buffers, salts, reducing agents, and accessory proteins, such as an RNase inhibitor or inorganic pyrophosphatase, may also be provided in the buffer, as well as the RNA polymerase. Combinations of buffers, salts, reducing agents, accessory proteins, RNA polymerase, and ribonucleotide triphosphates may be provided as part of the buffer system.

### Allosteric Transcription Factor

An allosteric transcription factor (aTF) regulates the in vitro transcription reaction in response to the binding of an analyte of interest. In an aspect, the transcription factor's activity is regulated by an analyte, e.g., a chemical or element in the sample. The allosteric transcription factor may bind or release an operator upon binding of an analyte of interest to the allosteric transcription factor.

In one embodiment, the assay may comprise two or more different allosteric transcription factors. In an example embodiment, a first aTF binds a first target chemical and binds to or releases from an operator of interest, and a second aTF binds to an operator of interest. In an embodiment, the operator of interest is the same, and the first and second aTF are different, e.g., conditionally activated by different binding moieties or conditions, e.g., sample pH. In an embodiment, the first aTF binds to a first operator and the second aTF binds to a second operator. The first and second operator can be disposed on the same or different reaction template.

In an embodiment, the first and second aTF are designed for binding of two different analytes of interest, and the first and second aTF bind to two different reaction templates. Thus, two different Trap nucleic acids can be transcribed from a first and second reaction temple. The lateral flow assay is then designed with a first and second capture region capable of specifically binding a first and second transduction module, respectively. An additional capture region for a control is provided with the capture regions configured for the binding of the transduction modules.

In one embodiment, the allosteric transcription factor is engineered to alter its ligand sensitivity and/or specificity or other intrinsic physical property. For example, such engineering may be desirable to tune the characteristics of the assay for detection in a particular range of concentration of an analyte.

In one embodiment, the transcription factor is a member of the AraC, AsnC/Lrp, Crp-Fnr, Fur, CadC/ArsR, Ic1R, TetR, Lad, MerR, CsoR, MarR or similar family. Further AraC, see, Lee et al., PNAS Dec. 1, 1987 84 (24) 8814-8818; doi: 10.1073/pnas.84.24.8814; AsnC/Lrp, see, Thaw et al., Nucleic Acids Research Mar. 1, 2006 34 (5) 1439-1449; doi: 10.1093/nar/gk1009; Crp-Fnr, see, Kõrner et al., December 2003, FEMS Microbiology Reviews, 27(5), 559-592, doi: 10.1016/S0168-6445(03)00066-4; Fur, see, Pohl et al., 2003 Molecular Microbiology 47(4), 903*915; doi: 10.1046/j.1365-2958.2003.03337.x; Ic1R, see, Yamamoto et al., Dec. 18, 2002, Molecular Microbiology, 47(1) 183-194, doi: 10.1046/j.1365-2958.2003.03287.x; Lad, see Gilbert et al., PNAS Dec. 1, 1966 56 (6) 1891-1898, doi: 10.1073/pnas.56.6.1891; MerR, see, Brown et al., June 2003, FEMS Microbiology Reviews, 27, (2-3), Pages 145-163, doi: 10.1016/S0168-6445(03)00051-2; MarR, see, Deochand and Grace, Jul. 3, 2017, Critical Reviews in Biochemistry and Molecular Biology, 52(6) 595-613, doi: 10.1080/10409238.2017.1344612; ArsR/CadC, see, Busenlehner et al, June 2003, FEMS Microbiology Reviews, 27(2-3), 131-143, doi: 0.1016/S0168-6445(03)00054-8; and TetR, see, Cuthbertson et al., Microbiology and Molecular Biology Reviews, 77(3) doi: 10.1128NIMBR.00018-13, specifically Tables 1 and 2 and their teachings of families of signal transduction systems and the TetR family of regulator, each of the references cited in this paragraph is incorporated herein by reference in its entirety.

In an embodiment, the transcription factor is selected from Table 1 and according to the analyte of interest to be detected.

TABLE 1
Allosteric Transcription Factors and Their Cognate Ligands

| Transcription Factor | Ligand |
|---|---|
| CadC | Lead, Cadmium |
| MerR | Mercury |
| CsoR | Copper |
| PbrR | Lead |
| TetR | Tetracyclines |
| LacI | Allolactose |
| ArsR | Arsenic |
| ChrB | Chromium |
| TtgR | Antibiotics, flavanols |
| Fur | Iron |

### Transduction Module

The cell-free biosensor lateral flow assay comprises a transduction module (FIG. 2). The transduction module is capable of detecting the Trap oligonucleotide that is the product of a sensor reaction when an analyte of interest is present (FIGS. 1, 3).

### Antibody-Reporter Conjugate

The transduction module can comprise an antibody-reporter conjugate or other binding moiety-reporter conjugate. The antibody of the antibody-reporter conjugate is capable of binding the chemical moiety of the Prey nucleic acid. The antibody may be associated with the detectable reporter. In one embodiment, the chemical moiety of the Prey nucleic acid is fluorescein, and the antibody of the antibody reporter is an anti-FITC antibody that is conjugated to a gold nanoparticle. Other binding pairs can be utilized in the system, and typically comprise a binding pair wherein one binding agent of the binding pair is conjugated to a detectable reporter and the other binding agent is a chemical moiety attached to the Prey nucleic acid. The binding partner of the binding agent attached to the Prey nucleic acid may also be comprised in a capture region of the lateral flow substrate, allowing accumulation and detection of the detectable reporter as a control for the reaction (FIGS. 2, 3).

### Reporter Molecule

In an example embodiment, the reporter molecule is a metal nanoparticle. The metal nanoparticle may be selected from the metals in groups IA, IB, IIB and IIIB of the periodic table, as well as the transition metals, especially those of group VIII. Preferred metals include gold, silver, aluminum, ruthenium, zinc, iron, nickel and calcium. Other suitable metals also include the following in all of their various oxidation states: lithium, sodium, magnesium, potassium, scandium, titanium, vanadium, chromium, manganese, cobalt, copper, gallium, strontium, niobium, molybdenum, palladium, indium, tin, tungsten, rhenium, platinum, and gadolinium. The metals are preferably provided in ionic form, derived from an appropriate metal compound, for example the A13+, Ru3+, Zn2+, Fe3+, Ni2+ and Ca2+ ions. See, e.g. Xu, Ning, Jin, Shuang and Wang, Li. “Metal nanoparticles-based nanoplatforms for colorimetric sensing: A review” Reviews in Analytical Chemistry, vol. 40, no. 1, 2020, pp. 1-11doi:10.1515/revac-2021-0122. In an embodiment, the detectable reporter molecule is a gold nanoparticle.

Further exemplary reporter molecules comprise a latex or carbon nanoparticle, or a colorimetric enzyme such as horseradish peroxidase, beta-galactosidase, or catecholase, or nanozymes, see, e.g. Calabria et al., Recent Advancements in Enzyme-Based Lateral Flow Immunoassays, Sensors 2021, 21, 3558; doi: 10.3390/s21103358.

### Bait Oligonucleotide

The Bait oligonucleotide may comprise a single stranded nucleic acid that may be further modified with a chemical moiety attached. In an aspect, the Bait nucleic acid is between about 25 and 50 nucleotides in length. In one embodiment, the bait nucleic acid is 50%, or greater similarity to sequence 5′-ACTACCGTCAGCATTATGTGAGTGAAACAA-3′ (SEQ ID NO: 1), for example, 50%, 55%, 60%, 65%, 65%, 70%, 75%, 80%, 85%, 90% 95%, 96%, 97%, 98% 99% sequence similarity. In an aspect, the Bait nucleic acid comprises the sequence 5′-ACTACCGTCAGCATTATGTGAGTGAAACAA-3′ (SEQ ID NO: 1).

The Bait nucleic acid may comprise a chemical moiety. In an embodiment, the moiety is capable of binding a molecule in a test zone capture region of the substrate (FIG. 3). In an aspect, the chemical moiety is biotin.

Bait oligonucleotides can be designed such that hairpins, homodimers, or heterodimers with the Prey oligonucleotide were unlikely to form using rational design and the RNAFold Web server (doi: 10.1093/nar/gkn188). The concentration of Bait oligonucleotide can range from 10 pM to 5 μM. Oligonucleotide concentrations can be optimized for use in the assay, for example by individually titrating concentrations of the Bait oligonucleotide and the Prey oligonucleotide so as to either minimize the amount of chemically synthesized oligonucleotide capable of being detected, to maximize the difference in regulated versus unregulated and/or induced vs uninduced in vitro transcription reaction test band densities. Further optimization of the system, for example, buffer components and reporter elements, can be optimized in a similar manner. DNA and protein components may vary in concentrations ranging from 1 pM to 100 μM, buffer components in concentrations ranging from 1 μM to 500 mM, antibody concentrations ranging from 0.1 to 10 mg/mL, and all other components may comprise concentrations ranging from 1 pM to 1 μM.

### Prey Oligonucleotide

The Prey oligonucleotide may comprise a single stranded nucleic acid that may be further modified with a chemical moiety attached. In an aspect, the Prey nucleic acid is between about 25 and 50 nucleotides in length. In one embodiment, the Prey nucleic acid is 50%, or greater similarity to sequence 5′-AGTGATATTGCCACCGACCTCAATCAATAA-3′ (SEQ ID NO: 2), for example, 50%, 55%, 60%, 65%, 65%, 70%, 75%, 80%, 85%, 90%, 95%, 96%, 97%, 98%, or 99% sequence similarity. In an aspect, the Prey nucleic acid comprises or consist of the sequence 5′-AGTGATATTGCCACCGACCTCAATCAATAA-3′ (SEQ ID NO: 2).

In another embodiment, the Prey nucleic acid is 50%, or greater similarity to sequence 5′-CTGACTCTCCTCTACTTCGTCTCGTATCAC-3′ (SEQ ID NO: 4), for example, 50%, 55%, 60%, 65%, 65%, 70%, 75%, 80%, 85%, 90%, 95%, 96%, 97%, 98%, or 99% sequence similarity. In an aspect, the Prey nucleic acid comprises or consists of the sequence 5′-CTGACTCTCCTCTACTTCGTCTCGTATCAC-3 (SEQ ID NO: 4).′

The Prey nucleic acid may comprise a chemical moiety. In an embodiment, the moiety is capable of binding a molecule in a test zone capture region of the substrate (FIG. 3). In an aspect, the chemical moiety is Fluorescein.

Prey oligonucleotides can be designed such that hairpins, homodimers, or heterodimers with the Bait oligonucleotide were unlikely to form using rational design and the RNAFold Web server (doi: 10.1093/nar/gkn188). The concentration of Prey oligonucleotide can range from 10 pM to 5 μM. Oligonucleotide concentrations can be optimized for use in the assay, for example by individually titrating concentrations of the Prey oligonucleotide and the Bait oligonucleotide so as to either minimize the amount of chemically synthesized oligonucleotide capable of being detected, to maximize the difference in regulated versus unregulated and/or induced vs uninduced in vitro transcription reaction test band densities. Further optimization of the system, for example, buffer components and reporter elements, can be optimized in a similar manner. DNA and protein components may vary in concentrations ranging from 1 pM to 100 buffer components in concentrations ranging from 1 μM to 500 mM, antibody concentrations ranging from 0.1 to 10 mg/mL, and all other components may comprise concentrations ranging from 1 pM to 1 μM.

### Capture Region

The lateral flow device substrate may comprise one, two, three or more capture regions or zones. Each capture region may comprise one or more binding agents. In an example embodiment, the lateral flow substrate comprises a first capture line, typically a horizontal line running across the device, but other configurations are possible (FIG. 2). A first binding agent that specifically binds a first molecule or binding agent is fixed or otherwise immobilized to the first capture region. A second capture region is located towards the opposite end of the lateral flow substrate downstream from the first binding region. A second binding agent is fixed or otherwise immobilized at the second capture region. The second binding agent specifically binds a detectable reporter. For example, the detectable reporter may be a particle, such as a colloidal particle, that when it aggregates can be detected visually. The particle may be modified with an antibody that specifically binds the second molecule or other binding agent.

### Binding Agent

Specific binding agents for use in the assays are designed for binding pairs, and the binding-integrating molecules comprise any members of binding pairs that can be used in the present invention. Such binding pairs are known to those skilled in the art and include, but are not limited to, antibody-antigen pairs, enzyme-substrate pairs, receptor-ligand pairs, and streptavidin-biotin. In addition to such known binding pairs, novel binding pairs may be specifically designed. A characteristic of binding pairs is the binding between the two members of the binding pair.

In certain example embodiments, a lateral flow device comprises a lateral flow substrate comprising a first end for application of a sample (i.e., a sample pad). In some cases, the lateral flow device also includes a conjugate pad, where detection reagents are stored. In other cases, the lateral flow device has only a conjugate pad to which the sample is directly applied. (FIG. 2).

The first region is loaded with a detectable ligand, such as those disclosed herein, for example a gold nanoparticle (FIG. 2). The gold nanoparticle may be modified with a first antibody, such as an anti-FITC antibody.

In one example embodiment, and for purposes of further illustration, a first capture region comprises streptavidin and a Bait nucleic acid comprises biotin (FIG. 2). It is also contemplated that the Bait nucleic acid may be included in the sample/conjugate pad, rather than in the capture region. When the presence of an analyte of interest is present and causes differential transcription of the Trap RNA, the Trap RNA forms a bridge with Bait nucleic acid and the Prey nucleic acid. The biotinylated Bait oligonucleotide associates with a streptavidin capture region of the substrate, and a fluorescein-labeled Prey nucleic acid is accumulated in the streptavidin capture region. The fluorescein-labeled Prey nucleic acid allows for enrichment of anti-FITC labeled gold nanoparticles, i.e., a detectable reporter, in the streptavidin capture region, forming a detectable signal at the capture region (FIGS. 1-3). Therefore, in the presence of one or more targets, the detectable reporter will accumulate at a first, test band, indicating the presence of the one or more targets (analytes of interest) in the sample. The first and second capture regions, e.g., a test capture region and a control capture region can be in any order on the substrate. In a preferred embodiment the test capture region is positioned before the control capture region on the substrate.

In another example embodiment, a first capture region comprises streptavidin and a Bait nucleic acid is complexed with a partially complementary fluorescein-labelled Prey nucleic acid. In a particular embodiment, the Trap RNA transcribed comprises greater complementarity to the Bait nucleic acid, for example at least 10%, 20%, 30%, 40%, 50% or more greater complementarity to the Bait nucleic acid. When the presence of an analyte of interest is present and causes differential transcription of the Trap RNA, the Trap RNA with greater complementarity to the Bait nucleic acid and displaces the Prey nucleic acid from the first capture region. The fluorescein-labelled Prey nucleic acid is not able to allow for enrichment of anti-FITC labeled gold nanoparticles, i.e., a detectable reporter, in the streptavidin capture region, and no detectable signal will arise at the capture region. Therefore, in the presence of one or more targets, the detectable reporter will not accumulate at the test band, indicating the presence of one or more targets (analytes of interest) in the sample. Accordingly, detection of the presence of one or more targets in the sample may be indicated by the absence of a signal at the test band.

### Substrate Materials

Substrates suitable for use in lateral flow assays are known in the art. A substrate may be a flexible materials substrate, for example, including, but not limited to, a paper substrate, a fabric substrate, or a flexible polymer-based substrate. These may include, but are not necessarily limited to membranes or pads made of cellulose and/or glass fiber, polyesters, nitrocellulose, or absorbent pads (Saudi Chem Soc 19(6):689-705; 2015).

Lateral support substrates may be located within a housing (see for example, “Rapid Lateral Flow Test Strips” Merck Millipore 2013). The housing may comprise at least one opening for loading samples and a second single opening or separate openings that allow for reading of detectable signal generated at the first and second capture regions.

### Sample Loading Zone

The sample loading zone is provided on a first end of the substrate (FIG. 2). The substrate may be exposed to the sample passively, by temporarily immersing the substrate in a fluid to be sampled, by applying a fluid to be tested to the substrate, or by contacting a surface to be tested with the substrate. Any means of introducing the sample to the substrate and in particular the sample loading zone may be used as appropriate. A first region may be loaded with one or more reagents or modules in accordance with the invention. For example, a first region, which may be the same or different as the sample loading zone, may comprise a sensor reaction module, detectable reporter, and/or transduction module reagents in the first region (FIG. 2). Alternatively, one or more of the sensor reaction module, detectable reporter, and/or transduction module reagents may be provided upon mixing of the sample with any of the reagents in an individual discrete volume such as an ampoule. In an aspect, the reagents are lyophilized and the sample is mixed with the lyophilized reagents and added to the lateral flow substrate at the time of assay. Advantageously, the reagents can be lyophilized for cold-chain independence and long-term storage, and readily reconstituted on the lateral flow substrate for field applications. One or more of the reagents may also be loaded onto the lateral flow assay with the sample loaded subsequent to loading of the reagents.

An individual discrete volume is a discrete volume or discrete space, such as a container, receptacle, or other defined volume or space that can be defined by properties that prevent and/or inhibit migration of nucleic acids and reagents necessary to carry out the methods disclosed herein, for example a volume or space defined by physical properties such as walls, for example the walls of a well, tube, or a surface of a droplet, which may be impermeable or semipermeable, or as defined by other means such as chemical, diffusion rate limited, electro-magnetic, or light illumination, or any combination thereof. Exemplary discrete volumes or spaces useful in the disclosed methods include microscope slides with regions defined by depositing reagents in ordered arrays or random patterns, tubes (such as, centrifuge tubes, microcentrifuge tubes, test tubes, cuvettes, conical tubes, and the like), bottles (such as glass bottles, plastic bottles, ceramic bottles, Erlenmeyer flasks, scintillation vials and the like), wells (such as wells in a plate), plates, pipettes, or pipette tips among others.

### Analyte of Interest

Analytes of interest that can be evaluated with the assays and methods disclosed herein include, but are not limited to, chemicals and molecule targets from environmental and biological samples. In an embodiment, the lateral flow device is designed for detection of a heavy metal. In an embodiment, the heavy metal is lead, cadmium, mercury, zinc, copper, manganese, chromium, cobalt, nickel, antimony, or thallium.

In an embodiment, the chemical is a pesticide or pesticide metabolite. For example, the pesticide may be atrazine, paraquat, a pyrethroid insecticide, 1,3,-dichloropropene, or an organophosphate.

In one embodiment, the chemical is an environmental contaminant. In an embodiment, the chemical is a PFOS or PFOA or another perfluorinated compound, a polychlorinated biphenyl, a dioxin, a bisphenol, or a phthalate.

The analyte of interest can comprise an antibiotic, or metabolite thereof. Exemplary antibiotics may be a tetracycline, an aminoglycoside, a carbapenem, a cephalosporin, a sulfonamide, a macrolide, a glycopeptide, a lincomycin, a penicillin, or a quinolone.

In an embodiment, the analyte of interest is a biological metabolite such as a sugar, hormone, neurotransmitter, lipid, or alcohol. For example, the metabolite may be glucose, cortisol, dopamine, serotonin, cholesterol, methanol, or ethanol.

### Samples

Samples for assay may be an environmental sample or biological sample that is a crude sample, which has not been purified or further manipulated prior to testing. In embodiments, a sample may be an environmental sample, such as water, soil, or a surface sample.

In an example embodiment, the sample is a water sample. Water samples may be from a drinking water sample from a home, educational facility, business, or other public or private place. The water sample may be an environmental source like a river, lake, pond, or groundwater, In an example embodiment, the water is from a containment pond, such as an ash pond.

In an example embodiment, the sample is from an industrial biotechnology source, or is a culture supernatant, cell lysate, or other input or output product from metabolic engineering.

### EXAMPLE METHODS AND ASSAYS

The low cost and adaptability of the assay platform lends itself to a number of applications including rapid and sensitive detection of target analytes of interest in environmental samples, including at relevant detection levels and multiplexed analyte detection. Methods of detection can comprise contacting a sample with the sensor module and transferring the sample to a first end of the lateral flow device comprising the sample loading portion, wherein the sample flows from the sample loading portion of the substrate towards a first and second capture regions, and generating a detectable signal (FIGS. 1-3). Exemplary methods of detection are detailed further in the Working Examples.

Methods may comprise incubating the sample for a period of time such that a detectable signal is generated. In an embodiment, the sample is incubated at room temperature. The sample may be incubated at a higher temperature, for example 30° C., 31° C., 32° C., 33° C., 34° C., 35° C., 36° C., 37° C., 38° C., 39° C. or 40° C. In an example embodiment, the reaction is run at a temperature above room temperature for a shorter period of time relative to a reaction performed at room temperature. The assay may be run for 5, 10, 15, 20 25, 30, 35, 40, 45, 50, 55 or 60 minutes.

Further embodiments are illustrated in the following Examples which are given for illustrative purposes only and are not intended to limit the scope of the invention.

### EXAMPLES

### Example 1—Lead Detection

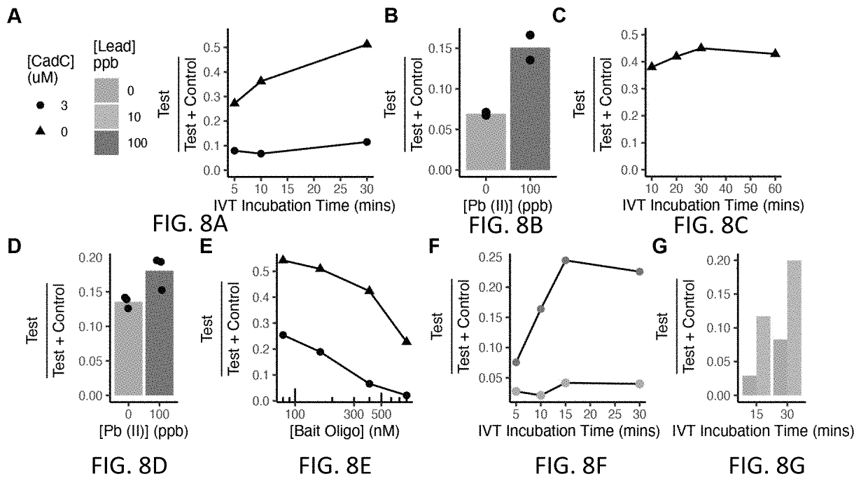

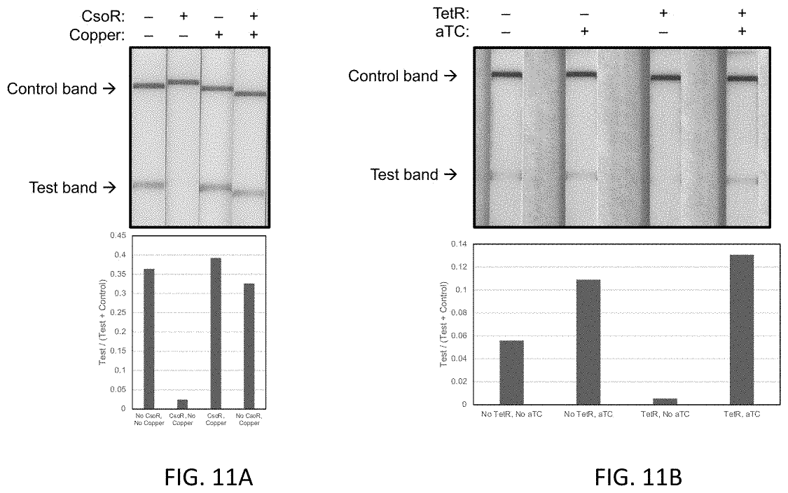

The examples describe development of an exemplary invention of a LFA-based visualization of cell-free biosensor read outs. In the system, an aTF regulates the expression of a “Trap” RNA which, is flowed through a LFA device with specifically designed “Bait” and “Prey” DNA oligos that detect the presence of the Trap RNA through binding interactions (FIGS. 1-3). Data validating the development of a lead (Pb) assay capable of detecting regulation-relevant concentrations of lead using the CadC aTF biosensor are shown (FIGS. 8-10). Additionally, considerations for optimization of the sensor are included and additional examples, demonstrating the extensibility of the system for other analytes of interest, are provided (FIG. 11). Importantly, this assay is fast, returning results visible by eye in under an hour, and can be run at room temperature, negating the need for heating elements and power.

The exemplary lateral flow system is comprised of two modules depicted in FIG. 1. The first is a sensing module consisting of an in vitro transcription (IVT) cell-free biosensor reaction in which an allosteric transcription factor (aTF) regulates a promoter for an RNA polymerase. In the presence of a ligand of interest, the aTF alters its interaction with the promoter region and allows transcription of an RNA termed a “Trap” RNA. This designed Trap RNA serves as an input to the downstream transduction module, which converts presence of the Trap into a visible output.

The transduction module has similarities to a sandwich-style ELISA in a lateral flow format, although it is distinct from previous sandwich-style ELISA formats. A designed biotinylated “Bait” DNA oligo associates with a streptavidin-enriched region of the lateral flow strip. Also in the solution is a designed fluorescein-labelled “Prey” DNA oligo with no complementarity to the Bait oligo. The Trap RNA transcribed by the sensing module has complementarity to the Prey oligo at its 5′ end and complementarity to the Bait oligo at its 3′ end and serves to bridge the two Bait and Prey oligos and form a ternary complex, allowing enrichment of the fluorescein-Prey at the test zone of the LFA. Anti-FITC antibody labelled gold nanoparticles then accumulate and are immobilized at the test zone, and a visible red band appears when Trap is present (FIGS. 1-3).

Proof of Concept of ssDNA and Purified ssRNA Trap Detection

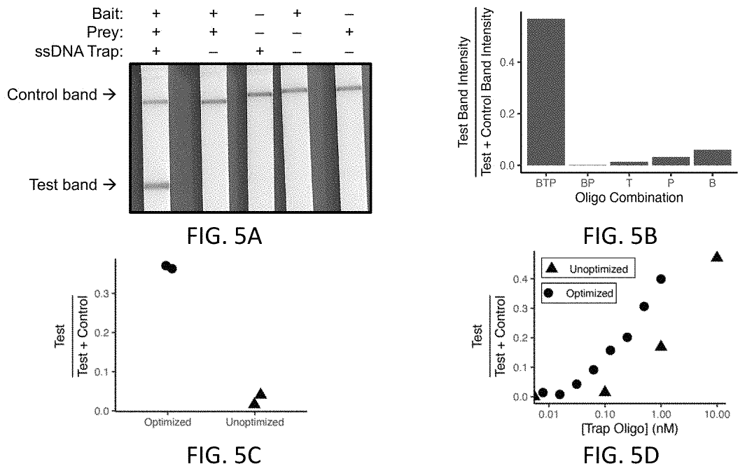

Before Applicants tested the ability to detect Trap RNA, Applicants tested the system's ability to detect the Trap in the form of a single-stranded DNA oligo. Bait, Trap DNA, and Prey ssDNA oligos were mixed combinatorially at a final concentration of 1.5 μM, vortexed briefly, then applied to a Milenia Biotec HybriDetect lateral flow strip with pictures taken periodically after application. Within a couple of minutes, a strong test band appeared on the strip exposed to Bait, Prey and Trap ssDNA oligos, but no intense bands appeared on any of the other strips. After approximately 10 minutes, the strips were removed from buffer and imaged against a dark background (FIG. 5A). To quantify band intensity, the central area of each strip was isolated, and the area under the curve of the test and control bands was calculated using ImageJ [https://doi org/10.1038/nmeth.2089, https://doi.org/10.1038/nmeth.2019]. To normalize across strips, test band intensity was divided by the sum of the test and control band intensities (FIG. 5B).

While successful in activating the LFA strip, it was reasoned that this initial 1.5 μM concentration of oligos may not be optimal. Specifically, it was reasoned that having excess amount of Bait oligo may be saturating the test zone, and excess mobile Bait may be sequestering ssDNA Trap oligo and preventing it from associating with stationary Bait in the test zone. To determine whether or not the amount of Bait and Prey oligo impacts assay sensitivity, an optimized assay formulation was developed consisting of 80 nM Bait and 3.2 nM Prey oligo. This was tested head-to-head against the unoptimized 1 μM Bait and Prey assay for the ability to detect 1 nM ssDNA Trap oligo. The optimized assay performed better than the unoptimized, with stronger band intensity (FIG. 5C). Applicants then performed a titration of ssDNA Trap oligo for the optimized and unoptimized sensors. The optimized sensor showed detection of ssDNA Trap oligo below 100 pM, though the unoptimized sensor failed to detect oligo at these levels (FIG. 5D).

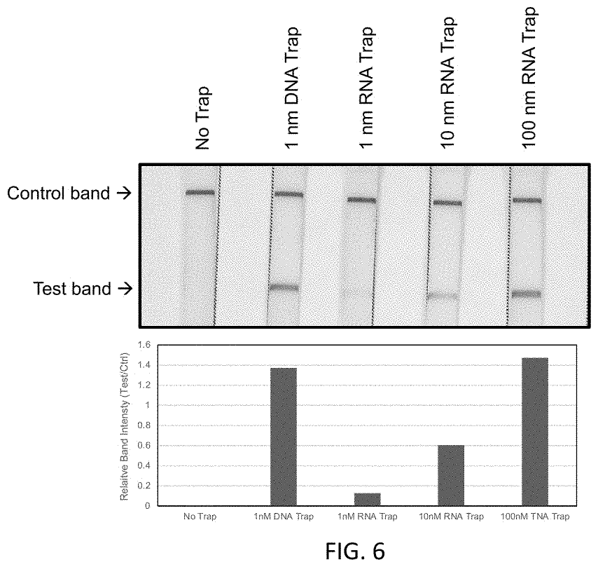

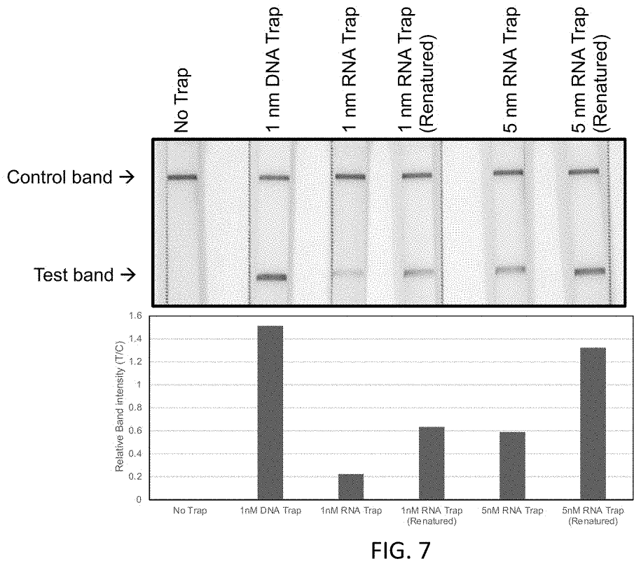

Having validated the function of the transduction module using a Trap DNA oligo, Applicants then tested the ability of the system to detect purified single-stranded Trap RNA. Applicants amplified a Trap RNA transcription template from a DNA plasmid encoding, from 5′ to 3′ end, a T7 RNA polymerase promoter (TAATACGACTCACTATA (SEQ ID NO: 5)), a short 5 base pair spacer to ensure robust transcription (GGAGG), a cadO operator sequence for binding by the CadC aTF (CTCAAATAAATATTTGAATGAAC (SEQ ID NO: 6)), the Trap sequence, and T7 terminator (TAGCATAACCCCTTGGGGCCTCTAAACGGGTCTTGAGGGGTTTTTTG (SEQ ID NO: 7)) as depicted in FIG. 4. Following PCR amplification, the product was purified using the Qiagen QIAquick PCR Purification Kit and its concentration determined using Invitrogen's Qubit dsDNA BR Assay Kit. 1 μg of PCR product was then added to an NEB HiScribe T7 High Yield RNA Synthesis Kit following manufacturer's instructions for small RNA and incubated for 4 to 6 hours at 37° C. The synthesis product was brought to 50 μL using nuclease free water and was then purified according to the NEB Monarch RNA Cleanup Kit. The purified RNA was then quantified using Invitrogen's Qubit ssRNA BR Assay Kit. Three concentrations of Trap RNA (1, 10, and 100 nM) and control reactions (containing either No Trap or 1 nM DNA Trap) were run in parallel with 80 nM Bait and 7 nM Prey. In brief, Bait and Prey, and Trap were added in Milenia HybriDetect's running buffer to create a master mix. Trap was then added to the individual aliquots of the master mix (except for the negative “No Trap” control, which was brought to final volume of 100 μL with additional running buffer) and then applied to the lateral flow strip for 10 minutes and then photographed. As the results show (FIG. 6), while Trap RNA was visually detectable on the lateral flow strip, there was a substantial difference in the intensity of the test band with the lowest concentration of Trap RNA (1 nM) resulting in a faint band. This difference in test band intensity is likely due in part to the reduced hybridization efficiency of a mixed RNA:DNA duplex but can be explained largely by the secondary structure of Trap RNA resulting in suboptimal folding. Applicants therefore performed an additional test with purified Trap RNA at 1 and 5 nM that included an additional heat denaturation step in which the samples were heated to 60° C. for 5 minutes and then cooled to room temperature in the presence of Bait and Prey before application to the lateral flow strip. As the results show (FIG. 7), performing a simple renaturation of the purified Trap RNA in the presence of Bait and Prey improved band intensity by a factor of 2.8 (for 1 nM Trap) and 2.4 (for 5 nM Trap). In these experiments, band intensity was quantified by isolating the central area of the strip, containing test and control regions, and the area under the curve of the test and control bands was calculated using ImageJ. Relative band intensity for each strip was then calculated as test band intensity divided by the control band intensity. These results indicate that folding of the Trap RNA is an important factor and can be used to tune the sensitivity and efficiency of the sensor.

Having validated the function of the transduction module using DNA oligos and purified RNA, Applicants then tested the ability of the system to detect Trap RNA expressed by T7 polymerase when immediately applied to the Transduction Module without purification. Applicants created an expression construct consisting of a T7 promoter with a downstream cadO operator site for the lead and cadmium sensitive CadC aTF, a promoter/operator combination which Applicants have previously shown to be functional in the ROSALIND biosensing system, and a further downstream sequence encoding a Trap RNA (FIG. 4). In vitro transcription (IVT) reactions were assembled by adding 25 nM template DNA and the lead-sensitive CadC aTF to reaction buffer (50 mM Tris HCl, 75 mM KCl, 12 mM MgCl₂, 10 mM dithiothreitol, 100 μg/mL bovine serum albumin, 0.3 U thermostable inorganic phosphatase, 30 ug/mL T7 RNA polymerase, and 20 mM each rATP, rCTP, rGTP, rUTP). For reactions including aTF, reaction was assembled without T7 RNA polymerase and incubated at room temperature for ten minutes before T7 RNA Polymerase was added.

Three 20 μL IVT reactions with or without 3 μM of purified CadC dimer were incubated at 37° C. for 5, 10, and 30 minutes. Upon reaction completion, samples were moved to an aluminum block chilled on ice and 1 μL of 0.5 M EDTA was added to samples to quench the reaction. When all samples were done, they were added to 80 μL of Tris-buffered saline with a final concentration of 80 nM ssDNA Bait and 3.2 nM ssDNA Prey oligos. The resulting mixture was then added to lateral flow strips as above. While the 0 μM CadC samples showed an increase in lateral flow test band density from 5 to 10 to 30 minutes, the 3 μM CadC samples did not increase in intensity until 30 minutes, and not nearly to the extent as the unregulated reaction (FIG. 8A). This result demonstrates that visualization of a regulated and unregulated IVT reaction are visible and compatible with the Transduction Module and lateral flow device.

Using Biosensor-Controlled Transcription Reactions to Detect Lead (Pb) with Lateral Flow Test Strips

Validating that the system was able to transcribe and detect Trap RNAs and that such transcription was regulatable with allosteric transcription factor biosensors, we then tested the system's ability to detect lead acetate at 100 parts per billion (ppb). Sensor reactions were assembled as above with 3 μM dimeric CadC, then water or lead was added to the IVT reaction and incubated at 37° C. for 10 minutes. Upon mixing with buffer containing 80 nM Bait and 3.2 nM Prey oligos and dipping a test strip in for 10 minutes, test band intensity was stronger for the 100 ppb lead samples than the no added lead samples (FIG. 8B), confirming that the combination of the sensor module and transduction module were able to detect the presence of lead and transduce it to a visible output on the lateral flow strip.

### Optimizing Reaction Conditions to Detect Lead Using LFAs and Room Temperature IVT Reactions

As phage-derived RNA polymerases have been shown to be active at lower temperatures (Krieg, 1990), and a major limitation of existing reporter-based aTF biosensors is the necessity of a heating element, Applicants decided to test the reactions at room temperature. Unregulated samples were prepared as above, and incubated at room temperature for 10, 20, 30, and 60 minutes. Even after 10 minutes, the test band intensity was significant and did not increase much past 20 minutes (FIG. 8C). Testing 100 ppb lead induction showed an increase in band intensity on addition of lead, though the change was small before later optimization (FIG. 8D).

Applicants reasoned the transduction module was overly optimized. No IVT reaction is completely leak-free, and a small amount of transcript can accumulate even with strong repression of the transcription reaction. To de-sensitize the transduction module, Applicants titrated in increasing amounts of Bait oligo to serve as a “sink” for Trap oligo. Increasing the amount of Bait to 800 nM per 10 μL reaction reduced the test band signal of regulated to near zero, while only reducing the intensity of the test band of unregulated reactions by −50% (FIG. 8E). Sensor reactions were then assembled with or without 100 ppb lead and incubated for 5, 10, 15, and 30 minutes and added them to buffer containing the re-optimized transduction module components. Detection of leak in the un-induced samples were greatly reduced by the excess Bait sink, while test bands of 100 ppb lead samples were strong and peaked quickly at 15 minutes (FIG. 8F). Applicants then tested our improved sensor's ability to detect the EPA regulatory limit of 15 ppb. A 15 minute transcription time was optimal, providing >2-fold increase in test band intensity (FIG. 8G).

Device Formats that Support Freeze-Drying and Simple Rehydration of Tests

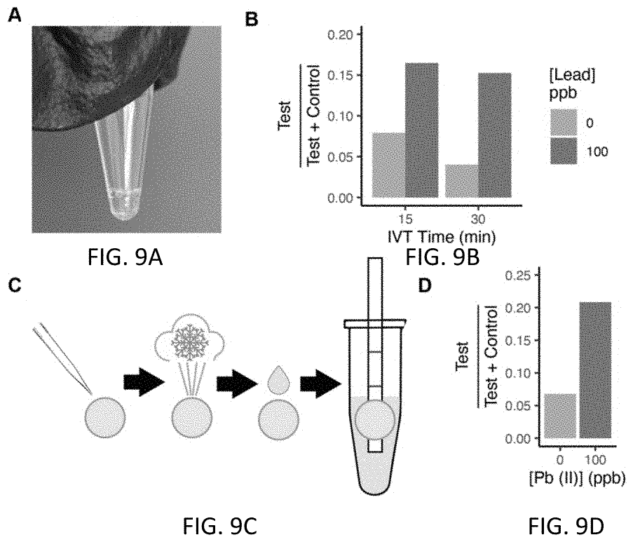

Next, a device embodiment was developed that allows tests to be freeze-dried and rehydrated for use. Applicants first lyophilized the sensor reactions in microcentrifuge tubes. Ten μL reactions were assembled with trehalose as a lyoprotectant and the improved Bait concentration, cooled to −80° C., and lyophilized overnight. The next morning, all water had sublimated and the dried reaction was present as rings at the 10 μL mark in the tubes (FIG. 9A). Samples were rehydrated with water containing 0 or 100 ppb lead, incubated at room temperature for 15 or 30 minutes, and applied to the LFA strips according to the procedure above. In both cases, test bands were stronger than the control bands (FIG. 9B). Next, Applicants tried lyophilizing the reaction onto paper disks to allow for simple rehydration and application to LFA strips for use (FIG. 9C). Twenty μL sensor reactions were deposited on 6 mm diameter Whatman Grade 1 paper disks that had been blocked with 0.4% BSA and lyophilized. Disks were rehydrated by adding twenty μL water with 0 or 100 ppb lead acetate. Disks were then incubated for fifteen minutes at room temperature, placed in buffer containing the improved transduction module composition, and applied to LFA strips. A stronger test band appeared for 100 ppb lead sample than the lead-free sample confirming that this device embodiment works as intended (FIG. 9D).

### Sensor Module Configuration to Tune Sensitivity

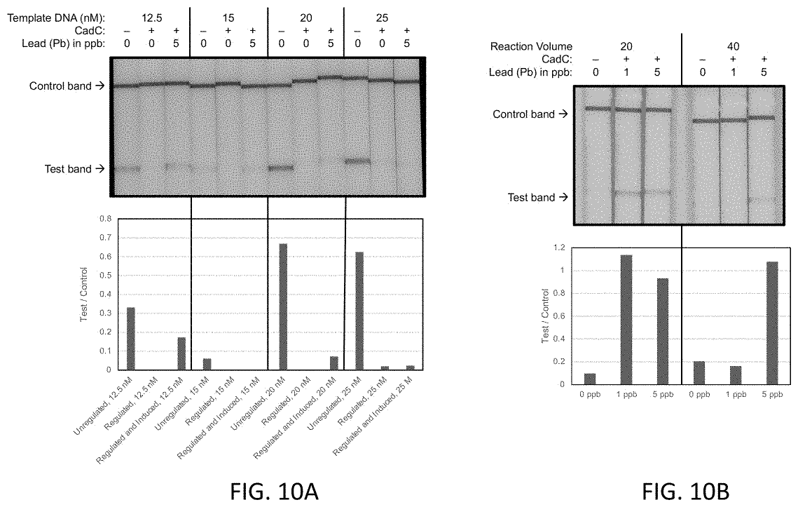

Next, certain parameters of the Sensor Module were altered to increase sensitivity of the test to 5 parts per billion lead in anticipation of updates to the regulatory limit. Applicants tested reactions as above, substituting the previously used 25 nM template DNA for 20, 15, and 12.5 nM template DNA. Reducing template DNA concentrations in the sensor module resulted in a decrease of unregulated reaction band intensity, but an increase in relative band intensity of sensor module exposed to 5 ppb lead (FIG. 10A). Next, the effective concentration of all sensor module components was decreased by increasing the amount of lead-containing water by 2-fold. At the standard 20 uL reaction volume, 1× sensor module concentration, 0 ppb lead failed to produce a visible test line, but both 1 and 5 ppb lead produced a visible test line (FIG. 10B). In contrast, 40 uL reaction volume, 0.5× sensor module concentration retained strong repression at 0 ppb lead, but only 5 ppb lead led to the appearance of a test band, showing that altering sensor module composition allows for tuning of test activation thresholds.

### Example 2—Copper Detection

To demonstrate the extensibility of the platform, Applicants created a sensor module capable of heavy metal copper ions using the CsoR allosteric transcription factor (UniProt ID P9WP49). Sensor reactions were set up 5 nM DNA template containing the T7 RNA polymerase promoter, csoO operator sequence (5′-GTAGCCCACCCCCAGTGGGGTGGGATAC-3′ (SEQ ID NO: 8)), Trap RNA sequence, and a T7 terminator. Sensor reactions were prepared with or without 0.5 μM purified CsoR transcription factor, and with or without 20.5 μM copper sulfate (corresponding to the EPA's regulatory limit for copper in drinking water). Bait and Prey concentrations were 80 nM and 7 nM, respectively. Following a 20-minute incubation at room temperature, samples were applied to the lateral flow strip and imaged. Control bands were visible in all 4 conditions and test bands were present in all samples containing copper and a control reaction which did not contain CsoR. Importantly, the test band was not present in the sample containing CsoR but not copper, validating the copper cell-free biosensor lateral flow assay (FIG.

### Example 3—Antibiotic Detection

To further demonstrate the extensibility of the platform beyond the detection of metals, Applicants built a cell-free biosensor lateral flow assay for the detection of tetracycline antibiotics, which are used in human and veterinary health, and in animal agriculture and aquaculture. A DNA template was constructed with a T7 promoter, the tetO operator sequence (TCCCTATCAGTGATAGAGA (SEQ ID NO: 9)), Trap RNA sequence, and T7 transcription terminator. 40 nM of template DNA was used in a sensor reaction with or without 2 μM TetR. After a 20-minute incubation with or without 2 μM anhydrotetracycline (aTC)—a synthetic tetracycline with improved stability—samples were contacted with the lateral flow strip and imaged after 10 minutes. The results show a clearly visible control band for all 4 samples. Test bands are faint, but visible, only in those samples containing aTC or lacking TetR. Importantly, the test band is present when TetR was included in the reaction along with aTC and not in the sample containing only TetR. Thus validating the cell-free biosensor lateral flow assay for tetracycline and showing the extensibility of the technology for chemical and molecular analytes of interest beyond metal ions. It reasons that any transcription factor that is compatible with ROSALIND technology can be adapted to function with the lateral flow readout.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A cell-free biosensor lateral flow device kit for the detection of one or more analytes of interest comprising:
a. a sensor reaction module comprising in vitro transcription reagents for an in vitro transcription reaction, wherein the sensor reaction module is conditionally activated by the presence of the analyte of interest to thereby produce a transcript;
b. a lateral flow device comprising a substrate with a sample deposition zone for receiving a sample, a first capture region comprising a first binding agent; and a second capture region comprising a second binding agent; and
c. a transduction module comprising oligonucleotides and a detectable reporter configured to interface the sensor reaction module with the lateral flow device.

2. The lateral flow device kit of claim 1, wherein one or more components of the transduction module and sensor reaction module are present or immobilized on the substrate.

3. The lateral flow device kit of claim 1, wherein the sensor reaction module reagents comprise a nucleic acid template, an allosteric transcription factor (aTF) capable of binding the analyte of interest, a buffer system enabling transcription, and a nucleic acid polymerase, or any combination thereof.

4. The lateral flow device kit of claim 3, wherein the buffer system comprises nucleotide triphosphates, buffers, salts, reducing agents, and accessory proteins.

5. The lateral flow device kit of claim 3, wherein the nucleic acid template comprises an operator sequence to which the aTF is capable of binding.

6. The lateral flow device kit of claim 1, wherein the transduction module further comprises a Bait nucleic acid, and a Prey nucleic acid.

7. The lateral flow device kit of claim 6, wherein the nucleic acid template encodes a polynucleotide comprising a 5′ end with reverse complementarity to the Prey nucleic acid and a 3′ end complementary to the Bait nucleic acid.

8. The lateral flow device kit of claim 6, wherein the Bait nucleic acid is a single stranded nucleic acid.

9. The lateral flow device kit of claim 6, wherein the Bait nucleic acid comprises at least 50% sequence similarity to 5′-ACTACCGTCAGCATTATGTGAGTGAAACAA-3′ (SEQ ID NO: 1).

10. The lateral flow device kit of claim 6, wherein the Bait nucleic acid comprises 5′-ACTACCGTCAGCATTATGTGAGTGAAACAA-3′ (SEQ ID NO: 1).

11. The lateral flow device kit of claim 6, wherein the Bait nucleic acid is between 25 and 50 nucleotides long.

12. The lateral flow device kit of claim 6, wherein the Bait nucleic acid comprises chemical moiety, optionally wherein the chemical moiety is biotin.

13. The lateral flow device kit of claim 6, wherein the Prey nucleic acid is a single stranded nucleic acid.

14. The lateral flow device kit of claim 6, wherein the Prey nucleic acid comprises at least 50% sequence similarity to 5′-AGTGATATTGCCACCGACCTCAATCAATAA-3′ (SEQ ID NO: 2).

15. The lateral flow device kit of claim 6, wherein the Prey nucleic acid comprises 5′-AGTGATATTGCCACCGACCTCAATCAATAA-3′ (SEQ ID NO: 2).

16. The lateral flow device kit of claim 6, wherein the Prey nucleic acid is between 25 and 50 nucleotides long.

17. The lateral flow device kit of claim 6, wherein the Prey nucleic acid comprises a chemical moiety, optionally wherein the chemical moiety is a fluorescein.

18. The lateral flow device kit of claim 6, wherein the detectable reporter comprises an antibody-reporter conjugate.

19. The lateral flow device kit of claim 18, wherein the antibody-reporter conjugate comprises an antibody that binds to a chemical moiety attached to the Prey nucleic acid conjugated to the detectable reporter.

20. The lateral flow device kit of claim 19, wherein the antibody is an anti-FITC antibody.

21. The lateral flow device kit of claim 1, wherein the detectable reporter comprises a gold, carbon or latex nanoparticle, or a colorimetric enzyme such as horseradish peroxidase, beta-galactosidase, or catecholase.

22. The lateral flow device kit of claim 1, wherein the in vitro transcription reagents comprise a linear or circular double stranded DNA molecule template.

23. The lateral flow device kit of claim 3, wherein the nucleic acid template is a linear or circular double stranded DNA molecule.

24. The lateral flow device kit of claim 3, wherein the nucleic acid polymerase is a DNA-dependent RNA polymerase.

25. The lateral flow device kit of claim 24, wherein the RNA polymerase is from a bacteriophage or a bacterium.

26. The lateral flow device kit of claim 24, wherein the RNA polymerase consists of a single sub-unit.

27. The lateral flow device kit of claim 24, wherein the RNA polymerase is T7 RNA polymerase, SP6 RNA polymerase, Syn5 RNA polymerase, KP34 RNA polymerase, SPβ RNA polymerase, VSW-3 RNA polymerase, or T3 RNA polymerase.

28. The lateral flow device kit of claim 25, wherein the RNA polymerase is E. coli RNA polymerase.

29. The lateral flow device kit of claim 1, wherein the transcription module comprises one or more nucleic acids, and the transcript produced has complementarity to one or more of the nucleic acids of the transcription module.

30. The lateral flow device kit of claim 3, wherein the allosteric transcription factor regulates the transcription reaction in response to the binding of the analyte of interest.

31. The lateral flow device kit of claim 3, wherein the allosteric transcription factor is a member of the AraC, AsnC/Lrp, Crp-Fnr, Fur, CadC/ArsR, Ic1R, TetR, LacI, MerR, CsoR or MarR family.

32. The lateral flow device kit of claim 3, wherein the allosteric transcription factor is engineered to alter its ligand sensitivity and/or specificity or other intrinsic physical property.

33. The lateral flow device kit of claim 3, wherein the allosteric transcription factor is regulated by a chemical or element in the sample.

34. The lateral flow device kit of claim 1, wherein the sensor module is applied to the sample deposition zone.

35. The lateral flow device kit of claim 1, wherein the sensor module is lyophilized, optionally wherein the sensor module is lyophilized in the sample deposition zone.

36. The lateral flow device kit of claim 1, wherein the analyte is a heavy metal.

37. The lateral flow device kit of claim 36, wherein the heavy metal is lead.

38. The lateral flow device kit of claim 36, wherein the heavy metal is copper.

39. The lateral flow device kit of claim 36, wherein the heavy metal is cadmium, mercury, zinc, manganese, chromium, cobalt, nickel, antimony, or thallium.

40. The lateral flow device kit of claim 1, wherein the analyte is a pesticide or pesticide metabolite.

41. The lateral flow device kit of claim 1, wherein the analyte is atrazine.

42. The lateral flow device kit of claim 1, wherein the analyte is the pesticide paraquat, a pyrethroid insecticide, 1,3,-dichloropropene, or an organophosphate.

43. The lateral flow device kit of claim 1, wherein the analyte is an environmental contaminant.

44. The lateral flow device kit of claim 1, wherein the analyte is an antibiotic.

45. The lateral flow device kit of claim 1, wherein the analyte is a tetracycline, an aminoglycoside, a carbapenem, a cephalosporin, a sulfonamide, a macrolide, a glycopeptide, a lincomycin, a penicillin, or a quinolone, or a metabolite thereof.

46. The lateral flow device kit of claim 1, wherein the analyte is a biological metabolite selected from a group consisting of a sugar, hormone, neurotransmitter, lipid, or alcohol.

47. The lateral flow device kit of claim 46, wherein the biological metabolite is glucose, cortisol, dopamine, serotonin, cholesterol, methanol, or ethanol.

48. The lateral flow device kit of claim 1, wherein the sample is water.

49. The lateral flow device kit of claim 1, wherein the sample is a drinking water sample from a home, educational facility, business, or other public or private place.

50. The lateral flow device kit of claim 1, wherein the sample is water from an environmental source selected from a group consisting of a river, lake, pond, or groundwater.

51. The lateral flow device kit of claim 1, wherein the sample is wastewater from a municipality, community, home, educational facility, factory, or other business or building.

52. The lateral flow device kit of claim 1, wherein the sample is from an industrial biotechnology source.

53. The lateral flow device kit of claim 1, wherein the sample is a biological sample, optionally saliva, urine, plasma or blood.

54. The lateral flow device kit of claim 1, wherein the sample is culture supernatant, cell lysate, or other input or output product from a metabolic engineering.

55. The lateral flow device kit of claim 3, comprising two or more allosteric transcription factors.

56. The lateral flow device kit of claim 55, wherein a first and second aTF bind to different target molecules or chemicals.

57. The lateral flow device kit of claim 55, wherein the aTF bind to the same operator on the template.

58. The lateral flow device kit of claim 55, wherein the aTF bind to a different operator on the template.

59. A method for detecting an analyte of interest in a sample, comprising contacting the sample with the sensor reaction module of the kit and transferring the sample to the sample loading zone of the lateral flow device according to claim 1, wherein the sample flows from the sample loading portion of the substrate towards the first and second capture regions and generates a detectable signal.


SEQUENCE LISTING (9 sequences)

SEQ ID NO: 1 - DNA, 30 bp, synthetic construct
  source 1..30: mol_type = other DNA
actaccgtcagcattatgtgagtgaaacaa

SEQ ID NO: 2 - DNA, 30 bp, synthetic construct
  source 1..30: mol_type = other DNA
agtgatattgccaccgacctcaatcaataa

SEQ ID NO: 3 - DNA, 155 bp, synthetic construct
  source 1..155: mol_type = other DNA
taatacgactcactataggaggctcaaataaatatttgaatgaacggttattgattgaggtcggtggcaatatcactttgtttcactcacataatgctgacggtagtgtagcataaccccttggggcctctaaacgggtcttgaggggttttttg

SEQ ID NO: 4 - DNA, 30 bp, synthetic construct
  source 1..30: mol_type = other DNA
ctgactctcctctacttcgtctcgtatcac

SEQ ID NO: 5 - DNA, 17 bp, synthetic construct
  source 1..17: mol_type = other DNA
taatacgactcactata

SEQ ID NO: 6 - DNA, 23 bp, synthetic construct
  source 1..23: mol_type = other DNA
ctcaaataaatatttgaatgaac

SEQ ID NO: 7 - DNA, 47 bp, synthetic construct
  source 1..47: mol_type = other DNA
tagcataaccccttggggcctctaaacgggtcttgaggggttttttg

SEQ ID NO: 8 - DNA, 28 bp, synthetic construct
  source 1..28: mol_type = other DNA
gtagcccacccccagtggggtgggatac

SEQ ID NO: 9 - DNA, 19 bp, synthetic construct
  source 1..19: mol_type = other DNA
tccctatcagtgatagaga